(19) United States
(12) Patent Application Publication
(10) Pub. No.: US 2010/0303880 A1
(43) Pub. Date: Dec. 2, 2010

(54) Tissue scaffolding comprising surface folds for tissue engineering

(76) Inventors: Harry K. Reddy, Lexington, MA (US); Joannis V. Yannas, Newton, MA (US); Brendan Harley, Cambridge, MA (US); Christopher J. Zagorski, Essex (GB)

Correspondence Address: Pearl Cohen Zedek Latzer, LLP, 1500 Broadway, 12th Floor, New York, NY, 10036, US

(21) Appl. No.: 11/919,494

(22) PCT Filed: Apr. 17, 2006
(86) PCT No.: PCT/US2006/014484

§ 371 Date: Jul. 12, 2010

Related U.S. Application Data
(60) Provisional application No. 60/675,481, filed on Apr. 28, 2005.

(51) Int. Cl. A61F 2/00; A61K 35/12; A61P 17/02; A61P 17/00; B28B 1/14

(52) U.S. Cl. 424/423; 424/93.7; 264/299

(57) ABSTRACT

The invention is directed to solid gradient scaffolds, methods of producing the same, and therapeutic applications arising from their utilization. Specifically, the gradient scaffolding includes, inter-alia, surface folds of various configuration for increasing surface area to volume of the scaffold.

### FIELD OF INVENTION

This invention relates to large scale scaffolding and methods of producing the same. Specifically, the scaffolding includes, inter-alia, surface folds of various configuration for increasing a surface area to volume ratio of the scaffold.

### BACKGROUND OF THE INVENTION

Implanting a scaffold to regenerate lost or damaged tissue requires the use of a scaffold that supports adequate cell migration into and around the scaffold, short-term support of these cells following implantation with an adequate supply of oxygen and nutrients and long-term angiogenesis and remodeling of the scaffold (degradation of the scaffold and remodeling of the vasculature and tissue architecture). If all these functions are not supported, new stroma will not be formed and tissue regeneration will not occur.

Scaffolds are prefabricated supports, which may be seeded with cells. While cells can easily adsorb into the outermost portions of the scaffold, cell distributions may not be uniform throughout the scaffold due to random motility and limitations in the diffusion of nutrients. This in turn may lead to uneven and distorted regeneration of tissue, which, if allowed to persist, may create other pathologies. Even if cells are homogenously distributed throughout a large-scale scaffold, there is a need for a vascular supply to nourish the cells in the interior of the scaffold, since these cells are positioned in a location within the scaffold, which is not readily accessible to the surrounding vasculature and are therefore deprived of nutrients and oxygen necessary for their long term viability. Cell survival necessitates being within the diffusion distance of a capillary, for the formation of a concentration gradient facilitating an exchange whereby the cell can receive an adequate concentration of oxygen and nutrients. While a vascular supply can grow into an implanted scaffold from surrounding vascularized tissue, the angiogenic process takes time, which may result in cell death in the scaffolding interior, prior to adequate vascularization.

One of the limitations to date in successful tissue engineering is a lack of an appropriate material and architecture whereby complex tissues may be assembled, in particular providing the ability of appropriate cells to align themselves along desired characteristic dimension to form a functioning tissue. Current methodology also is lacking in terms of providing an appropriate substrate that facilitates formation of tissue for regions of tissue attached to each other, where each region differs in terms of its resident cell type and composition. Additionally, there are no scaffolds which can provide all of the above at the size/scale large enough to be applicable for use to regenerate tissue in the breast or other large organs which have large characteristic dimensions (>1 cm).

Therefore there is a need for a scaffold capable of supporting tissue regeneration on a large scale where the scaffold facilitates infiltration of vasculature to support cells located below the surface of the scaffold within a period short enough to ensure viability of these cells, as well as supports adequate cell migration into and around the scaffold, short-term support of the cells immediately following implantation with an adequate supply of oxygen and nutrients and long-term angiogenesis and remodeling of the scaffold.

### SUMMARY OF THE INVENTION

In one embodiment, the invention provides a solid, biocompatible scaffold for, implantation, comprising surface folds, wherein said surface folds increase said scaffold surface area to volume ratio (SA/V), by at least 20%.

In another embodiment, this invention provides a process for preparing a solid scaffold comprising surface folds, the process comprising the steps of:
applying a polymeric suspension to a mold comprised of a conductive material, wherein a surface of said mold which is in contact with said suspension has numerous folds; and
subjecting the suspension-filled mold in (a) to conditions whereby said suspension is solidified;
   whereby removal of said mold exposes said solid scaffold comprising surface folds.

In another embodiment, this invention provides a method of organ or tissue engineering in a subject, comprising the step of implanting a scaffold of this invention in a subject.

In one embodiment, the methods and scaffold of the invention are used to facilitate or accelerate tissue repair or regeneration, including in one embodiment, in application in wound healing.

### DETAILED DESCRIPTION OF THE INVENTION

The invention is directed to solid gradient scaffolds, methods of producing the same, and therapeutic applications arising from their utilization.

A large three-dimensional shape has a significantly lower surface area to volume ratio compared with typical scaffolds produced to regenerate skin (thin sheets) and peripheral nerves (small tubes); since the infiltration of blood vessels into a scaffold occurs from the vasculature surrounding the surface, it would be difficult to maintain cell viability in cells disposed below a few millimeters from the scaffold's outer surface, since penetration may be insufficient. A lower surface area to volume ratio would also impede the migration of cells into the scaffold from the surrounding tissue. A scaffold with increased surface area to volume ratio (SA/V) is thus desirable, which is provided, in one embodiment, with the scaffolds of this invention, which provide for sufficient angiogenesis to occur in time to support the viability of cells in the interior of the scaffold and so that, in another embodiment, migration of cells into the scaffold from the surrounding tissue is not impeded.

Tissue engineering, repair and regeneration has been significantly hampered to date by a lack of appropriate material and architecture whereby complex tissue may be assembled, in particular providing the ability of appropriate cells, including multiple cell types, to align themselves in three dimensions to form functioning tissue.

In one embodiment, the invention provides a solid, biocompatible scaffold for implantation, comprising surface folds, wherein said surface folds increase said scaffold surface area to volume ratio (SA/V), by at least 20%.

In one embodiment, the term “scaffold” or “scaffolds” refers to three-dimensional structures that assist in the tissue regeneration process by providing a site for cells to attach, proliferate, differentiate and synthesize an extracellular matrix, eventually leading to tissue remodeling.

In one embodiment, the term “gradient scaffold”, refers to a scaffold that is comprised of a material which varies throughout the scaffold, in terms of in one embodiment, the concentration of components of which the scaffold is comprised, or in another embodiment, its porosity (which may be reflected in other embodiments in terms of, pore size, pore shape, percent porosity, tortuosity, interconnectivity), or in another embodiment, its cross-link density, or in another embodiment, its density. In another embodiment, the term “gradient scaffold” refers to scaffold comprised of material with varying pore diameter throughout the scaffold.

In another embodiment, the term “biocompatible” refers to products that break down into elements that are beneficial or in another embodiment, not harmful to the subject or his/its environment. In another embodiment, the term “biocompatible” refers to and tends not to induce fibrosis, inflammatory response, host rejection response, cell adhesion or combination thereof, following exposure of the scaffold to a subject or cell in said subject. In another embodiment, the term “biocompatible” refers to any substance or compound that has minimal (i.e., no significant difference is seen compared to a control), if any, effect on surrounding cells or tissue exposed to the scaffold in a direct or indirect manner.

In one embodiment, the term “biocompatible” refers to a material which, when placed in a biological tissue, does not provoke a toxic response. In one embodiment, the material which is biocompatible may be organic or in another embodiment synthetic. In one embodiment, the biocompatible material may treat, or in one embodiment, replaces, or in another embodiment augment, or in another embodiment stimulates or repairs any biological tissue.

In one embodiment, the invention provides a scaffold comprising a biocompatible material, which, in another embodiment may be carbohydrate, or in another embodiment, single amino acid, or in another embodiment, a monomer of a biocompatible polymer as described herein, or in another embodiment, a combination thereof.

In one embodiment, the invention provides a scaffold comprising at least one polymer, wherein, in another embodiment the polymer is a synthetic, or in another embodiment a natural polymer, or in another embodiment a ceramic, or in another embodiment a metal, or in another embodiment an extracellular matrix protein, or in another embodiment an analogue thereof, or in another embodiment, a combination thereof.

In another embodiment, the polymers of this invention may be copolymers. In another embodiment, the polymers of this invention may be homo- or, in another embodiment heteropolymers. In another embodiment, the polymers of this invention are synthetic, or, in another embodiment, the polymers are natural polymers. In another embodiment, the polymers of this invention are free radical random copolymers, or, in another embodiment, graft copolymers. In one embodiment, the polymers may comprise proteins, peptides or nucleic acids.

In another embodiment, the polymers may comprise biopolymers such as, for example, collagen. In another embodiment, the polymers may comprise biocompatible polymers such as polyesters of [alpha]-hydroxycarboxylic acids, such as poly(L-lactide) (PLLA) and polyglycolide (PGA); poly-p-dioxanone (PDO); polycaprolactone (PCL); polyvinyl alcohol (PVA); polyethylene oxide (PEO); polymers disclosed in U.S. Pat. Nos. 6,333,029 and 6,355,699; and any other bioresorbable and biocompatible polymer, co-polymer or mixture of polymers or co-polymers described herein.

In one embodiment, biodegradable polymers are usually based on functional groups such as esters, anhydrides, orthoesters, and amides. Rapidly biodegradable polymers include in another embodiment poly[lactide-co-glycolide], polyanhydrides, and polyorthoesters. In one embodiment, bioerodible polymers include polylactides, polyglycolides, and copolymers thereof, poly(ethylene terephthalate), poly(butyric acid), poly(valeric acid), poly(lactide-co-caprolactone), poly(lactide-co-glycolide), polyanhydrides, polyphosphazenes, poly(.epsilon.-caprolactone), poly(dioxanone), poly(hydroxybutyrate), poly(hydroxyvalerate), polyorthoesters, blends, and copolymers thereof.

Examples of biodegradable and biocompatible polymers of acrylic and methacrylic acids or esters include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), etc. Other polymers which can be used in the present invention include polyalkylenes such as polyethylene and polypropylene; polyarylalkylenes such as polystyrene; poly(alkylene glycols) such as poly(ethylene glycol); poly(alkylene oxides) such as poly(ethylene oxide); and poly(alkylene terephthalates) such as poly(ethylene terephthalate). Additionally, polyvinyl polymers can be used which include polyvinyl alcohols, polyvinyl ethers, polyvinyl esters, and polyvinyl halides. Exemplary polyvinyl polymers include poly(vinyl acetate), polyvinyl phenol, and polyvinylpyrrolidone. Mixtures of two or more of the above polymers could also be used in the present invention.

In another embodiment, the polymers comprising extracellular matrix components may be purified from tissue, by means well known in the art. For example, if collagen is desired, in one embodiment, the naturally occurring extracellular matrix can be treated to remove substantially all materials other than collagen. The purification may be carried out to substantially remove glycoproteins, glycosaminoglycans, proteoglycans, lipids, non-collagenous proteins and nucleic acid (DNA or RNA), by known methods.

In one embodiment, the polymer may comprise Type I collagen, Type II collagen, Type IV collagen, gelatin, agarose, cell-contracted collagen containing proteoglycans, glycosaminoglycans or glycoproteins, fibronectin, laminin, elastin, fibrin, synthetic polymeric fibers made of poly-acids such as polylactic, polyglycolic or polyamino acids, polycaprolactones, polyamino acids, polypeptide gel, copolymers thereof and/or combinations thereof. In one embodiment, the scaffold will be made of such materials so as to be biodegradable.

In another embodiment, the solid polymers of this invention may be inorganic, and comprise for example, hydroxyapatite, all calcium phosphates, alpha-tricalcium phosphate, beta-tricalcium phosphate, calcium carbonate, barium carbonate, calcium sulfate, barium sulfate, polymorphs of calcium phosphate, ceramic particles, or combinations thereof.

In one embodiment, the polymers may comprise a functional group, which enables linkage formation with other molecules of interest, some examples of which are provided further hereinbelow. In one embodiment, the functional group is one which is suitable for hydrogen bonding (e.g., hydroxyl groups, amino groups, ether linkages, carboxylic acids and esters, and the like).

In another embodiment, functional groups may comprise an organic acid group. In one embodiment, the term “organic acid group” is meant to include any groupings which contain an organic acidic ionizable hydrogen, such as carboxylic and sulfonic acid groups. The expression “organic acid functional groups” is meant to include any groups which function in a similar manner to organic acid groups under the reaction conditions, for instance metal salts of such acid groups, such as, for example alkali metal salts like lithium, sodium and potassium salts, or alkaline earth metal salts like calcium or magnesium salts, or quaternary amine salts of such acid groups, such as, for example quaternary ammonium salts.

In one embodiment, functional groups may comprise acid-hydrolyzable bonds including ortho-ester or amide groups. In another embodiment, functional groups may comprise base-hydrolyzable bonds including alpha-ester or anhydride groups. In another embodiment, functional groups may comprise both acid- or base-hydrolyzable bonds including carbonate, ester, or iminocarbonate groups. In another embodiment, functional groups may comprise labile bonds, which are known in the art and can be readily employed in the methods/processes and scaffolds described herein (see, e.g. Peterson et al., Biochem. Biophys. Res. Comm. 200(3): 1586-159 (1994) 1 and Freel et al., J. Med. Chem. 43: 4319-4327 (2000)).

In another embodiment, the scaffold further comprises a pH-modifying compound. In one embodiment, the term “pH-modifying” refers to an ability of the compound to change the pH of an aqueous environment when the compound is placed in or dissolved in that environment. The pH-modifying compound, in another embodiment, is capable of accelerating the hydrolysis of the hydrolyzable bonds in the polymer upon exposure of the polymer to moisture and/or heat. In one embodiment, the pH-modifying compound is substantially water-insoluble. Suitable substantially water-insoluble pH-modifying compounds may include substantially water-insoluble acids and bases. Inorganic and organic acids or bases may be used, in other embodiments.

In one embodiment, the extracellular matrix proteins comprise a collagen, a glycosaminoglycan, or a combination thereof.

In one embodiment, as described herein, other molecules may be incorporated within the scaffold, which may, in another embodiment, be attached via a functional group, as herein described. In another embodiment, the molecule is conjugated directly to the scaffold.

In another embodiment, the polymers of this invention may comprise extracellular matrix components, such as hyaluronic acid and/or its salts, such as sodium hyaluronate; glycosaminoglycans such as dermatan sulfate, heparan sulfate, chondroiton sulfate and/or keratan sulfate; mucinous glycoproteins (e.g., lubricin), vitronectin, tribonectins, surface-active phospholipids, rooster comb hyaluronate. In some embodiments, the extracellular matrix components may be obtained from commercial sources, such as ARTHREASE™ high molecular weight sodium hyaluronate; SYNVISC® Hylan G-F 20; HYLAGAN® sodium hyaluronate; HEALON® sodium hyaluronate and SIGMA® chondroitin 6-sulfate.

In another embodiment, one or more biomolecules may be incorporated in the scaffold. The biomolecules may comprise, in other embodiments, drugs, hormones, antibiotics, antimicrobial substances, dyes, radioactive substances, fluorescent substances, silicone elastomers, acetal, polyurethanes, radiopaque filaments or substances, anti-bacterial substances, chemicals or agents, including any combinations thereof. The substances may be used to enhance treatment effects, reduce the potential for implantable article erosion or rejection by the body, enhance visualization, indicate proper orientation, resist infection, promote healing, increase softness or any other desirable effect.

In one embodiment, the scaffold varies in terms of its polymer concentration, or concentration of and component of the scaffold, including biomolecules and/or cells incorporated within the scaffold.

In one embodiment, the biomolecule may comprise chemotactic agents; antibiotics, steroidal or non-steroidal analgesics, anti-inflammatories, immunosuppressants, anti-cancer drugs, various proteins (e.g., short chain peptides, bone morphogenic proteins, glycoprotein and lipoprotein); cell attachment mediators; biologically active ligands; integrin binding sequence; ligands; various growth and/or differentiation agents (e.g., epidermal growth factor, IGF-I, IGF-II, TGF-β I-III, growth and differentiation factors, vascular endothelial growth factors, fibroblast growth factors, platelet derived growth factors, insulin derived growth factor and transforming growth factors, parathyroid hormone, parathyroid hormone related peptide, bFGF; TGF superfamily factors; BMP-2; BMP-4; BMP-6; BMP-12; sonic hedgehog; GDF5; GDF6; GDF8; PDGF); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments, DNA plasmids, or any combination thereof.

In one embodiment, the scaffold varies in terms of its polymer concentration, or concentration of and component of the scaffold, including biomolecules and/or cells incorporated within the scaffold.

In another embodiment, the scaffold may comprise one or more of an autograft, an allograft and a xenograft of any tissue with respect to the subject.

In one embodiment, the scaffolds may comprise cells. In one embodiment, the cells may include one or more of the following: chondrocytes; fibrochondrocytes; osteocytes; osteoblasts; osteoclasts; synoviocytes; bone marrow cells; mesenchymal cells; stromal cells; stem cells; embryonic stem cells; precursor cells derived from adipose tissue; peripheral blood progenitor cells; stem cells isolated from adult tissue; genetically transformed cells; a combination of chondrocytes and other cells; a combination of osteocytes and other cells; a combination of synoviocytes and other cells; a combination of bone marrow cells and other cells; a combination of mesenchymal cells and other cells; a combination of stromal cells and other cells; a combination of stem cells and other cells; a combination of embryonic stem cells and other cells; a combination of precursor cells isolated from adult tissue and other cells; a combination of peripheral blood progenitor cells and other cells; a combination of stem cells isolated from adult tissue and other cells; and a combination of genetically transformed cells and other cells.

In one embodiment, the scaffolds of this invention comprise surface folds, wherein said surface folds vary in one embodiment in terms of depth, which may range in another embodiment, from about 1 mm to about 10 cm, and in one embodiment, in terms of diameter, which may range in another embodiment from about 1 mm to about 5 cm, or in another embodiment, a combination thereof, within said scaffold. In one embodiment, the ratio between the length of the scaffold and the height of the scaffold is larger than 10.

In one embodiment the surface folds vary in depth from about 1 mm to 1 cm, or in another embodiment from about 1 cm to 2 cm, or in another embodiment from about 2 cm to 3 cm, or in another embodiment from about 3 cm to 4 cm, or in another embodiment from about 4 cm to 5 cm, or in another embodiment from about 5 cm to 6 cm, or in another embodiment from about 6 cm to 7 cm, or in another embodiment from about 7 cm to 8 cm, or in another embodiment from about 8 cm to 9 cm, or in another embodiment from about 9 cm-to 10 cm.

In one embodiment surface folds increase the surface area of the scaffold from about 15 to 300%, or in another embodiment, from about 15 to 50%, or in another embodiment, from about 50 to 100%, or in another embodiment, from about 100 to 150%, or in another embodiment, from about 150 to 200%, or in another embodiment, from about 200 to 250%, or in another embodiment, from about 250 to 300%.

In one embodiment, determination of surface area to volume ratio (SA/V) is done by any appropriate method known in the art, such as for example by using sorption isotherms (e.g. http://itl.chem.ufl.edu/4411L_f00/ads/sample/knight.html, or Gregg, S. J. and K. S. W. Sing (1982). Adsorption, Surface Area and Porosity. Academic Press, Inc., London, 2nd edition.), or in another embodiment, using modified glass beads method (see e.g. Jiahua Zhou, Milford A. Hanna (2004) Extrusion of Starch Acetate with Mixed Blowing Agents Starke, (2004) Vol 56, (10), 484-494).

In another embodiment, the invention provides a solid scaffold, in which the surface folds form a channel, where, in another embodiment, the channels are randomly distributed on the face of the scaffold, or in another embodiment, oriented along an axis, which in another embodiment, the width of said channel is greater at a point more proximal to the scaffold surface, than to its core. In one embodiment, the channel narrows from the periphery of the scaffold to its center. In another embodiment, the folds are randomly distributed throughout said scaffold.

In one embodiment the surface fold geometry is designed for a particular tissue formation, such as in one embodiment for regeneration of intestine tissue, or in another embodiment for kidney, or in another embodiment for bone, or in another embodiment for breast, or in another embodiment innervated tissue.

In one embodiment, the width of the channel varies between about 1 mm to 5 cm, or in another embodiment between about 1 mm to 1 cm, or in another embodiment between about 1 cm to 2 cm, or in another embodiment from about 2 cm to 3 cm, or in another embodiment from about 3 cm to 4 cm, or in another embodiment from about 4 cm to 5 cm.

In one embodiment, the invention provides a scaffold containing surface folds, wherein the scaffold further comprises cells, extracellular matrix components, growth factors, cytokines, hormones, inflammatory stimuli, angiogenic factors, or a combination thereof

In another embodiment, the scaffold is non-uniformly porous. In one embodiment, the term “porous” refers to a substrate that comprises holes or voids, rendering the material permeable. In one embodiment, non-uniformly porous scaffolds allow for permeability at some regions, and not others, within the scaffold, or in another embodiment, the extent of permeability differs within the scaffold.

In one embodiment, the pores within the scaffold are of a non-uniform average diameter. In another embodiment, the average diameter of said pores varies as a function of its spatial organization in said scaffold, or in another embodiment, average diameter of said pores varies as a function of the pore size distribution along an arbitrary axis of said scaffold.

In one embodiment, scaffolds that are non-uniformly porous are especially suited for tissue engineering, repair or regeneration, wherein the tissue is a connector tissue, or wherein the scaffold is utilized to engineer, repair or regenerate two or three, or more, tissues in close proximity to one another. A difference in porosity may facilitate migration of different cell types to the appropriate regions of the scaffold, in one embodiment. In another embodiment, a difference in porosity may facilitate development of appropriate cell-to-cell connections among the cell types comprising the scaffold, required for appropriate structuring of the developing/repairing/regenerating tissue. For example, dendrites or cell processes extension may be accommodated more appropriately via the varied porosity of the scaffolding material. In another embodiment, the permeability differences in the scaffolding material may prevent and enhance protein penetrance, wherein penetration is a function of molecular size, such that the lack of uniform porosity serves as a molecular sieve. It is to be understood that the gradient scaffolding of this invention may be used any purpose for which non-uniform porosity is desired, and is to be considered as part of this invention.

In another embodiment, the scaffold varies in its average pore diameter and/or distribution thereof. In another embodiment, the average diameter of the pores varies as a function of its spatial organization in said scaffold. In another embodiment, the average diameter of the pores varies as a function of the pore size distribution along an arbitrary axis of the scaffold. In another embodiment, the scaffold comprises regions devoid of pores. In another embodiment, the regions are impenetrable to molecules greater than 1000 Da in size.

In one embodiment, scaffolds that are non-uniformly porous, vary in their average pore diameter, which may range from 0.75 to 3000 μm, pore size distribution, which may range from about 20 to 200 μM, cross-link density, which may be modified by any crosslinking technology known in the art, or a combination thereof.

In one embodiment, average pore diameter may range between about 0.75 to about 3000 μM. In another embodiment, D_{3,2} is a measure of average pore diameter and in another embodiment follows a lognormal distribution. In one embodiment the term “D_{3,2}” refers to the average diameter of the pores calculated assuming spherical pores and inferring the average diameter from the surface area exposed to the measuring device.

In one embodiment, lognormal has the following frequency distribution:

$$df = \frac{1}{σ \sqrt{2 π}} exp [ - \frac{{( {d}_{p} - \overline{{d}_{p}} )}^{2}}{2 {σ}^{2}} ] {dd}_{p}$$

### Wherein:

dₚ is the pore diameter in μM
d_{p bar} is the average pore diameter
σ is the standard deviation of pore sizes in μM

In one embodiment, the size and shape of said scaffold is a function of the tissue into which the scaffold is to be implanted.

In another embodiment, the scaffold, when implanted, promotes angiogenesis within, or proximal to the scaffold.

In one embodiment the scaffold is comprised of a material whose stiffness is sufficient to resist compressive forces of tissue proximal to a site of implantation. In another embodiment, degree of cross-linking of the scaffold material is adjusted to compensate for the compressive forces of the surrounding tissue. In one embodiment, initial polymer slurry concentration is varied as a function of the compressive force of the target tissue. In one embodiment, plasticizers are embedded in the scaffold to allow imparting some elasticity to the scaffold, without collapse of the folds in the surface of the scaffold, which, in another embodiment will vary in depth and width depending on the compressive force of the surrounding target tissue.

Hydrophilic materials, both of a monomeric and a polymeric nature either exist in one embodiment as or in another embodiment can be converted into amorphous states which exhibit the glass/rubber transitions characteristic of amorphous macromolecules. These materials have well defined glass transition temperatures Tg which depend in one embodiment on the molecular weight or in another embodiment on the molecular complexity of the glass forming substance. Tg is depressed by the addition of diluents. Water is the universal plasticiser for all such hydrophilic materials. Therefore, the glass/rubber transition temperature is adjustable by in one embodiment the addition of water or an aqueous solution, or in another embodiment, the removal of water or an aqueous solution.

In one embodiment, the scaffold is fabricated using a process that creates an amorphous glassy-state solid, comprised of a biocompatible polymer. In one embodiment “glassy-state solid” refers to an amorphous metastable solid wherein rapid removal of a plasticizer causes increase in viscosity of the to biopolymer to the point where translational mobility of the critical polymer segment length is arrested and alignment corresponding to the polymer's inherent adiabatic expansion coefficient is discontinued.

In another embodiment, the plasticizer may be any substance of molecular weight lower than that of the biocompatible polymer that creates an increase in the free interstitial volume. In one embodiment, the plasticizer is an organic compound, which in one embodiment is triglyceride of varying chain length, or in another embodiment, the plasticizer is water.

In one embodiment, amorphous glassy-state solid is accomplished by rapid cooling of an aerated melt of the biocompatible polymer, or in another embodiment by rapid solvent removal under vacuum, or in another embodiment, by freeze-drying. In one embodiment, amorphous glassy-state solid is accomplished by extrusion, which in one embodiment is at temperatures higher than 65° C. or, in another embodiment, at temperatures between about 4 and about 40° C. In one embodiment, width, length, depth, or a combination thereof, of the surface folds are designed into the dye used for extrusion, in conjunction with extrusion conditions. It will be understood by a skilled person in the art, that any process capable of producing amorphous glass with high portion of interconnected porosity (sponge-like) where pore size is controllable by varying the fabrication conditions is appropriate for use for producing a scaffold of this invention and is thus within the scope of the invention.

In one embodiment, scaffolds are prepared according to the processes of this invention, in a highly porous form, by freeze-drying and sublimating the material. This can be accomplished by any number of means well known to one skilled in the art, such as, for example, that disclosed in U.S. Pat. No. 4,522,753 to Dagalakis, et al. For examples, porous gradient scaffolds may be accomplished by lyophilization. In one embodiment, extracellular matrix material may be suspended in a liquid. The suspension is then frozen and subsequently lyophilized. Freezing the suspension causes the formation of ice crystals from the liquid. These ice crystals are then sublimed under vacuum during the lyophilization process thereby leaving interstices in the material in the spaces previously occupied by the ice crystals. The material density and pore size of the resultant scaffold may be varied by controlling, in other embodiments, the rate of freezing of the suspension and/or the amount of water in which the extracellular matrix material is suspended at the initiation of the freezing process.

According to this aspect of the invention and in one embodiment, to produce scaffolds having a relatively large, uniform pore size and a relatively low material density, the extracellular matrix suspension may be frozen at a slow, controlled rate (e.g., −1° C./min or less) to a temperature of about −20° C., followed by lyophilization of the resultant mass. To produce scaffolds having a relatively small uniform pore size and a relatively high material density, the extracellular matrix material may be tightly compacted by centrifuging the material to remove a portion of the liquid (e.g., water) in a substantially uniform manner prior to freezing. Thereafter, the resultant mass of extracellular matrix material is flash-frozen using liquid nitrogen followed by lyophilization of the mass. To produce scaffolds having a moderate uniform pore size and a moderate material density, the extracellular matrix material is frozen at a relatively fast rate (e.g., >−1° C./min) to a temperature in the range of −20 to −40° C. followed by lyophilization of the mass.

In another embodiment, this invention provides a process for preparing a solid scaffold comprising surface folds, the process comprising the steps of applying a polymeric suspension to a mold comprised of a conductive material, wherein a surface of said mold which is in contact with said suspension has numerous folds; and subjecting the suspension-filled mold to conditions whereby said suspension is solidified, whereby removal of said mold exposes said solid scaffold comprising surface folds.

In one embodiment, “polymeric suspension” or “suspension” refers to any suspended system that would form a solid scaffold upon removal of one phase in the system. In one embodiment, the suspended system is a suspension, or in another embodiment emulsion, or in another embodiment, gel or in another embodiment, foam, or in another embodiment a thermodynamically incompatible polymer mixture. In one embodiment, the polymeric suspension is comprised of monomers or in another embodiment, single biocompatible molecules.

In one embodiment, the invention provides a process for preparing a solid scaffold comprising surface folds, wherein the process further comprises super-cooling the suspension-filled mold in after applying a polymeric suspension to a mold, at a constant temperature, for a period of time until said suspension is solidified, whereby ice crystals are formed in said solidified suspension, said crystals being oriented perpendicular to an edge of said scaffold. In another embodiment, the solidified suspension or a portion thereof, is exposed to conditions which enable sublimation in the exposed region, where in another embodiment, pores are formed perpendicular to an edge of said scaffold.

In one embodiment, the scaffold is comprised of surface folds, wherein said surface folds increase the surface area of the scaffold. In one embodiment, the folds may be coordinated in 3-D to form “tunnels” which may be oriented in another embodiment from the periphery to the core of the scaffold, such that in one embodiment, the diameter of the tunnel narrows as a function of the distance from the periphery. In another embodiment the increase in surface area is the result of removal of mold components creating open tunnels leading from the periphery of the scaffold into the center of the scaffold. In one embodiment, the surface folds form channels within the surface of the scaffold, thereby increasing the surface area to volume ratio (SA/V) of the scaffold. In another embodiment, the surface folds have gradually narrower radius as a function of the distance from the scaffold's periphery. In one embodiment, the open tunnels in the scaffold have a narrower diameter as a function of the distance from the scaffold's periphery.

According to this aspect of the invention, and in one embodiment, in order to produce gradient scaffolding of this invention, the freezing rate is controlled, such that a thermal gradient is created within the scaffold, during its formation. For example, a slurry of interest comprising polymers as described and/or exemplified herein, may be inserted in a supercooled silicone oil bath, as described by Loree et al. (1989) Proc. 15^{th} Annual Northeast Bioeng. Conf., pp. 53-54). According to this aspect, in one embodiment, the container is only partially immersed, and is not completely submerged in the bath, such that a freezing front which travels up the length of the container is created, thereby creating a temperature gradient within the slurry.

According to this aspect of the invention, and in another embodiment, in order to produce gradient scaffolding of this invention, solar bath effect is used to control ice crystallization rate and size thereby controlling pore size in the lyophilized mass. In one embodiment, a solute is incorporated into the mass and a temperature gradient is induced by placing the pan containing the mass on a cold plate, which in one embodiment may be the freeze-dryer shelf, or in another embodiment a heat lamp may be placed on top of the pan. Since solubility is a function of temperature, a solute concentration gradient will result. In another embodiment, solute concentration affects the freezing temperature, resulting in different crystal size in a fixed freezing time, which, in a gradually concentrated solute will result in graduated porosity with pore size inversely proportional to the direction of increased solute concentration. In one embodiment, the solute comprises heterogeneous nucleation centers for water.

In one embodiment, the gradient is preserved by halting the freezing process prior to achieving thermodynamic equilibrium. The means for determining the time to achieving thermodynamic equilibrium in a slurry thus immersed, when in a container with a given geometry, will be readily understood by one skilled in the art. Upon achieving the desired temperature gradient, the slurry, in one embodiment, is removed from the bath and subjected to freeze-drying. Upon sublimation, the remaining material is the scaffolding comprising the polymer, with a gradient in its average pore diameter.

In another embodiment, a gradient in freezing rate of the scaffold is generated with the use of a graded thermal insulation layer between the container, which contains the scaffold components, and a shelf in a freezer on which the container is placed. In one embodiment, a gradient in the thermal insulation layer is constructed via any number of means, well known in the art, such as, for example, the construction of a thicker region in the layer along a particular direction, or in another embodiment, by varying thermal conductivity in the layer. The latter may be accomplished via use of, for example, aluminum and copper, or plexiglass and aluminum, and others, all of which represent embodiments of the present invention.

In one embodiment, the invention provides a scaffold prepared according to the process described herein.

In another embodiment, pores may be specifically collapsed in the resulting scaffold

In one embodiment, local pore collapse can be obtained by any means such as moisture, air, physical, or in another embodiment, any means that would exceed the glass transition temperature of the matrix. In another embodiment, the glass transition temperature, or the glass/rubber transition (Tg) refers to the temperature of onset of translational mobility of the critical segment length of the biocompatible material used to comprise the scaffold of the invention. In another embodiment, the biocompatible material is any polymer used to comprise the scaffold described herein. In one embodiment, Tg is a function of the molecular weight of the biocompatible material used to comprise the scaffold, or in another embodiment, Tg is a function of the moisture content of the matrix following the fabrication of the amorphous glassy matrix, which, in another embodiment, is exposed to environmental conditions of varying relative humidity to control the Tg of the matrix. In one embodiment, “pore collapse” refers to the process wherein the material comprising the scaffold, having surpassed Tg undergoes realignment of the molecules to achieve the thermodynamically corresponding volume. In another embodiment, conditions that would locally decrease Tg to below the storage temperature are achieved by locally increasing moisture content, or in another embodiment by locally increasing temperature, or in another embodiment by locally incorporating plasticizers, or in another embodiment by decreasing the critical segment length of the material of the scaffold, which in one embodiment may be done through chemical reactions, or in another embodiment, enzymatically.

In another embodiment, the process further comprises the step of exposing the scaffold to a gradient of solutions, which are increased in their concentration of an enzyme, which degrades or solubilizes at least one extracellular matrix component. According to this aspect of the invention, and in one embodiment, digestion of at least one extracellular matrix component increases as a function of increasing enzyme concentration.

In one embodiment, the step of locally decreasing Tg to below that of the storage temperature, is followed by a change in environmental condition, increasing Tg such that the scaffold's Tg is above the storage temperature. In another embodiment, increasing Tg to above the storage conditions is achieved by dehydration of the matrix, which in one embodiment is done by exposing the matrix to temperatures lower than the desired Tg, or in another embodiment, by exposing the matrix to saturated salt solutions. In one embodiment, the saturated salt solution used is Lithium Chloride (LiCl), or in another embodiment Potassium Acetate (K⁺CH₃COO⁻) or in another embodiment to Phosphorous Pentoxide (P₂O₅), or in another embodiment to a concentration of Sulfuric acid imparting relative humidity values of below 0.35. In one embodiment, when exceeding Tg is achieved by locally heating the scaffold, removal of the heating element will result in local cooling of the scaffold material to below Tg, thereby inhibiting further pore collapse according to the methods of the invention. In one embodiment, increasing Tg may involve cross-linking of the scaffold material, thereby increasing the critical segment length (x).

In one embodiment, controlled pore collapse is conducted along an axis of the scaffold. In one embodiment, water evaporation from regions of interest may be accomplished at appropriate pressure known in the art, such as, for example, through the use of hot air directed at the region. According to this aspect of the invention, the dried regions will be devoid of pores, or in another embodiment, will be diminished in terms of the extent of porosity in the region, by the controlled collapse of these pores, due to surface tension issues. In one embodiment, the term degrade/s or solubilizes encompasses partial degradation or solubilization, or in another embodiment, complete degradation or solubilization.

In one embodiment, the enzyme is a collagenase, a glycosidase, or a combination thereof. In one embodiment, the enzyme is an endoglycosidase, which catalyzes the cleavage of a glycosidic linkage. In one embodiment, the endoglycosidase is a Heparitinase, such as, for example Heparitinase I, II or III. In another embodiment, the endoglycosidase is a Glycuronidase, such as, for example, Δ^{4,5}-Glycuronidase. In another embodiment, the glycosidase is an endo-xylosidase, endo-galactosidase, N-glycosidase or an endo-glucuronidase.

In one embodiment, the enzymes are purified, or in another embodiment, from recombinant sources. In one embodiment, the enzyme concentration is at a range between 0.001-500 U/ml. In another embodiment, the enzyme concentration is at a range between 0.001-500 U/ml, or in another embodiment, enzyme concentration is at a range between 0.001-1 U/ml, or in another embodiment, enzyme concentration is at a range between 0.001-10 U/ml, or in another embodiment, enzyme concentration is at a range between 0.01-10 U/ml, or in another embodiment, enzyme concentration is at a range between 0.01-100 U/ml, or in another embodiment, enzyme concentration is at a range between 0.1-10 U/ml, or in another embodiment, enzyme concentration is at a range between 0.1-100 U/ml, or in another embodiment, enzyme concentration is at a range between 1-10 U/ml, or in another embodiment, enzyme concentration is at a range between 1-100 U/ml, or in another embodiment, enzyme concentration is at a range between 10-100 U/ml, or in another embodiment, enzyme concentration is at a range between 10-250 U/ml, or in another embodiment, enzyme concentration is at a range between 10-500 U/ml, or in another embodiment, enzyme concentration is at a range between 100-500 U/ml or in another embodiment, enzyme concentration is at a range between 100-250 U/ml or in another embodiment, enzyme concentration is at a range between 50-100 U/ml or in another embodiment, enzyme concentration is at a range between 50-250 U/ml or in another embodiment, enzyme concentration is at a range between 50-500 U/ml.

In one embodiment, enzyme activity may be determined by any means well known to one skilled in the art. In one embodiment, enzyme degradation of a GAG may be determined by mass spectroscopy, to proton and carbon ¹³NMR analysis, or in another embodiment, capillary HPLC-ESI-TOF-MS, high performance liquid chromatography (HPLC), conventional chromatography, gel electrophoresis and the like.

According to this aspect of the invention, and in one embodiment, a gradient scaffold may be prepared by producing a scaffold comprised of a polymer, which is a copolymer, with a specific composition, and in a controlled manner, digesting or solubilizing at least one component of the scaffold, along a particular axis, or according to a desired geometry, thereby producing the gradient scaffold.

In one embodiment, a graft copolymer of two different extracellular matrix components is formed, such as for example a type I collagen and GAG. The final ratio of collagen/GAG may be equal, in another embodiment, to any combination between 85/15 to 100/0 w/w by methods well known in the art (Yannas, et al. PNAS 1989, 86:933)). According to this aspect of the invention, and in one embodiment, a length of the polymer is then exposed to a concentration gradient of a collagenase, for a period of time, wherein time, in another embodiment, is varied, which may, in another embodiment, provide for greater digestion of for example collagen, in some sections of the scaffold thus exposed. In one embodiment, digestion is a function of enzyme concentration, or in another embodiment, exposure time to a given concentration, or in another embodiment, a combination thereof.

Such controlled pore closure may be used for creating scaffolding, in another embodiment, for applications where biological baffles are useful. In one embodiment, the term “biological baffles” refers to matter, which physically isolates a biological activity in one region from that in an area adjacent thereto.

In one embodiment, controlled pore closure scaffolds are useful in scaffolding seeded with cells, conferring a particular biological activity, such as described in U.S. Pat. No. 4,458,678 or U.S. Pat. No. 4,505,266. Biological baffles created by controlled pore closure, in one embodiment, creates regions devoid of cells, or, in another embodiment, impenetrable to cells, or in another embodiment, both. Such baffles, in some embodiments, may be useful in separating particular cell types, seeded in the scaffold, or in another embodiment, creating discrete milieu, in separated regions, each with a particular biochemical makeup, such as, for example, regions which vary in terms of the types and/or concentration of cytokines, growth factors, chemokines, etc.

According to this aspect of the invention, and in one embodiment, the process further comprises the step of exposing the scaffold to a gradient of solutions, which are increased in their salt concentration. In another embodiment, the process further comprises the step of exposing the scaffold to a gradient of solutions, which are increased in their concentration of an enzyme, which degrades or solubilizes at least one extracellular matrix component. In another embodiment, the process further comprises the step of exposing the scaffold to a temperature gradient resulting in the creation of a gradient in crosslink density in the scaffold. In another embodiment, the process further comprises the step of exposing the scaffold to a gradient of solutions, which are increased in their concentration of cross-linking agent.

The scaffold, in one embodiment, is freeze-dried and sublimated, producing a porous material with a uniform composition, throughout the volume of the solid. In one embodiment, the solid is then exposed to an increasing salt gradient, such as, NaH₂PO₄, or, in another embodiment, NaCl, or in another embodiment, an electrolyte, or in another embodiment, combinations thereof (see for example, Yannas et al., JBMR, 14:107-131, 1980).

In one embodiment, the salt solution is at a range corresponding to an ionic strength of between 0.001 and 10. In another embodiment, the salt solution is at a range corresponding to an ionic strength of between 0.001 and 1, or in another embodiment, the salt solution is at a range corresponding to an ionic strength of between 0.01 and 10, or in another embodiment, the salt solution is at a range corresponding to an ionic strength of between 0.1 and 10, or in another embodiment, the salt solution is at a range corresponding to an ionic strength of between 1 and 10, or in another embodiment, the salt solution is at a range corresponding to an ionic strength of between 1 and 20, or in another embodiment, any range in concentration wherein selective solubilization is accomplished, while scaffold integrity is maintained.

According to this aspect of the invention, and in one embodiment, the process further comprises the step of exposing the scaffold to a gradient of solutions, which are increased in their concentration of an enzyme, which degrades or solubilizes at least one extracellular matrix component. In another embodiment, the process further comprises the step of exposing the scaffold to a temperature gradient. In another embodiment, the process further comprises the step of exposing the scaffold to a gradient of solutions, which are increased in their concentration of cross-linking agent.

According to this aspect of the invention, and in one embodiment, the process further comprises the step of exposing the scaffold to a temperature gradient, or in another embodiment to solutions with cross-linking agent gradient.

According to this aspect of the invention, and in one embodiment, the gradient scaffold produced may be further influenced by controlling the chemical composition of the resulting scaffold. In one embodiment, chemical composition may be controlled by a variation of methods described in U.S. Pat. No. 4,280,954.

For example, and in one embodiment, the scaffold is comprised of a graft copolymer of a type I collagen and a GAG, whose ratio is controlled by adjusting the mass of the macromolecules mixed to form the copolymer.

In one embodiment, the invention provides a method of organ or tissue engineering in a subject, comprising the step of implanting a scaffold of any of the embodiments mentioned herein.

In another embodiment, this invention provides a method of organ or tissue repair or regeneration in a subject, comprising the step of implanting a scaffold of this invention in a subject.

According to these aspects of the invention, and in one embodiment, the scaffold may be one produced by a process of this invention.

In one embodiment, use of the scaffolds for repair, regeneration of tissue is in cases where native tissue is damaged, in one embodiment, by trauma, or in another embodiment, compounded by diabetes. In another embodiment, the gradient scaffold allows for incorporation of individual cells, which are desired to be present in the developing/repairing/regenerating tissue.

According to these aspects of the invention, and in one embodiment, the method further comprises the step of implanting cells in the subject. In one embodiment, the cells are seeded on said scaffold, or in another embodiment, on the periphery of the scaffold. In another embodiment, the cells are stem or progenitor cells. In another embodiment, the method further comprises the step of administering cytokines, growth factors, hormones or a combination thereof to the subject. In another embodiment, the engineered organ or tissue is comprised of heterogeneous cell types. In one embodiment, the tissue is breast tissue, or in another embodiment skin tissue.

As can be seen from the forgoing description, the concepts of the present disclosure provide numerous advantages. For example, the concepts of the present disclosure provide for the fabrication of an implantable gradient scaffold, which may have varying mechanical properties to fit the needs of a given scaffold design. For instance, the pore size and the material density may be varied to produce a scaffold having a desired mechanical configuration. In particular, such variation of the pore size and the material density of the scaffold is particularly useful when designing a scaffold which provides for a desired amount of cellular migration therethrough, while also providing a desired amount of structural rigidity. In addition, according to the concepts of the present disclosure, implantable devices can be produced that not only have the appropriate physical microstructure to enable desired cellular activity upon implantation, but also has the biochemistry (collagens, growth factors, glycosaminoglycans, etc.) naturally found in tissues where the scaffolding is implanted for applications such as, for example, tissue repair or regeneration.

In one embodiment, the method of the invention is used for wound healing.

In one embodiment, the term “wound” refers to damaged biological tissue in the most general sense.

In another embodiment, the wound is a laceration of the skin. In one embodiments, the wound may be an abrasion of the skin with two separated parts of tissue which in another embodiment, need to be brought together. In one embodiments, the wound may refer to a surgical incision. In another embodiment, the wound may involve damage to lung tissue, arterial walls, or other organs with elastic fibers. In one embodiment, the wound may involve an abscess, or in another embodiment, the wound may be exacerbated by diabetes. In one embodiment, the methods and scaffolds of the invention are used to accelerate wound healing.

According to this aspect of the invention and in one embodiment, wound healing is divided into several overlapping phases. These include in one embodiment fibrin clot formation, recruitment of inflammatory cells, reepitheliazation, and matrix formation and remodeling. In one embodiment, the scaffold of the invention is comprised of invaginated surface topography, allowing for regrowth of disrupted blood vessels. In another embodiment, the scaffold further comprises cytokines and growth hormones, facilitating healing.

In one embodiment, neutrophils and monocytes are recruited to the site of injury by a number of chemotactic signals including in another embodiment the growth factors and cytokines released by the degranulating platelets, formyl methionyl peptides cleaved from bacterial proteins, and the by-products of proteolysis of fibrin and other matrix proteins. Neutrophil infiltration ceases in one embodiment after a few days, but macrophages continue to accumulate by continued recruitment of monocytes to the wound site. Activated macrophages release growth factors and cytokines thereby amplifying the earlier signals from the degranulating platelets.

In one embodiment, the gradient scaffold of the invention is seeded with epidermal cells at the periphery of the scaffold and implanted into the wound, thereby accelerating healing of the wound. According to this aspect of the invention and in one embodiment, a solid, porous, biocompatible gradient scaffold, seeded with epidermal cells and further comprising one or more extracellular matrix components or analogs thereof, is used to heal an open wound by implanting the scaffold into the wound, wherein the scaffold comprises elastin, neutrophils, monocytes and EGF. In another embodiment, the scaffold is additionally seeded with stem cells, which in one embodiment are engineered to express growth factors.

In one embodiment, the method and solid gradient scaffold of the invention is used for regeneration of breast tissue, following breast augmentation procedure. In another embodiment, following the incision and insertion of the gradient scaffold of the invention, inflammatory exudate starts to flow into the large open pore channels at the scaffold surface within the first few hours following implantation. Fibrin, formed from fibrinogen condenses creating a network on which blood vessels can grow. Other factors and cells present in exudate help reconstruct the stroma within the scaffold and promote angiogenesis. The vasculature in the pre-existing tissue becomes closer to the scaffold due to contraction of the surrounding tissue and increased pressure from the space taken up by the implant. An additional vascular network is also formed surrounding the scaffold as capsule forms. The high concentration of angiogenic factors in exudate and from migrating/seeded cells causes blood vessels to grow into the scaffold, supporting the nearby cells indefinitely.

The following examples are presented in order to more fully illustrate the preferred embodiments of to the invention. They should in no way be construed, however, as limiting the broad scope of the invention

### EXAMPLES

### Example 1

### Regeneration of a Large 3D Volume of Breast Tissue

The scaffold used is a sphere which is 50 mm in diameter, the pore structure form open channels at or near the surface which extend to the center of the scaffold. The diameter of these channels increases from the center to the scaffold surface, with the diameter near the surface as high as a few millimeters. As the channels extend toward the center they may divide to form a network of channels inside the scaffold, mimicking the progressive division of blood vessels in tissue. The scaffold is seeded with appropriate cells in the periphery. The cells extend from the outer surface to an approximate depth of 10 mm inside the scaffold. The scaffold also has VEGF bound onto the collagen fibers. The diameter of the pore channels at the scaffold surface is 1 mm.

The procedure for implanting the device is analogous to the procedure used to implant saline-filled breast implants. The scaffold is inserted by using a trans-axillary approach. The device is placed above the pectoralis major muscle, as placement below would expose the scaffold to a different cellular environment to the tissue types being regenerated. Surgeons also believe that placement below pectoralis major reduces capsular contracture, utilized to help bring the vascular bed in close proximity with the scaffold surface. The patient is placed under general anesthetic. Following the axillary incision the surgeon creates a small pocket to insert the scaffold between the breast gland tissue and pectoralis major. The scaffold is inserted into the space formed and the incision is closed. Following surgery the patient wears a specially designed undergarment to protect the device from being dislodged and from excessive compressive force. Pain medications are utilized as necessary following surgery. Once in place, the pressure from the surrounding tissue brings the existing vasculature in contact with the device's outer surface, forcing tissue into the invagination. The formation of capsule around the implant occurs spontaneously, creating multiple layers of fibrous tissue containing a variable amount of contractile cells, the innermost layers contain vasculature which is brought in close proximity to the scaffold. The degree to which capsule forms around the implant is dependant on the material from which it is composed, forming more around synthetic polymers.

Inflammatory exudate is released from capillaries in phases, bathing the wound in plasma proteins. Different cell types are recruited over different phases of time to remove damaged tissue, induce the formation of new tissue, reconstruct damaged matrix, basement membrane and connective tissue, and establish a new blood supply. Fluid exudate is released in three phases following injury: the first phase begins almost immediately after injury and involves a histamine-stimulated release of fluid and lasts anywhere between 8 to 30 minutes. The next phase is similar beginning straight after the first; it lasts longer, up to several days. The final phase commences a few hours after injury and the effects become maximal in 2-3 days, gradually resolving over a matter of weeks. Cellular exudate is produced in the second and third phases. The implanted scaffold allows the contents of the exudate to diffuse more easily and throughout the scaffold materials, but a sudden increase in tissue pressure doesn't occur. This will help exudate flow through the pores and channels in the scaffold, without a sudden increase in pressure damaging the implant. The components of exudate, both cellular and molecular (as detailed earlier) aid in angiogenesis and the regeneration of tissue.

The inflammatory exudate starts to flow into the large open pore channels at the scaffold surface within the first few hours following implantation. Fibrin, formed from fibrinogen condenses, creating a network on which blood vessels can grow. Other factors and cells present in exudate help reconstruct the stroma within the scaffold and promote angiogenesis. The vasculature in the pre-existing tissue comes closer to the scaffold due to contraction of the surrounding tissue and increased pressure from the space taken up by the implant. An additional vascular network is formed surrounding the scaffold as capsule forms. The high concentration of angiogenic factors in exudate and from migrating/seeded cells causes blood vessels to grow into the scaffold, supporting the nearby cells indefinitely.

The invaginations in the scaffolds structure decrease the distance blood vessels travel to reach the center of the scaffold. They also decrease the amount of blood vessel growth required to vascularize the outer regions of the scaffold, thus rapidly vascularizing a large proportion of the volume of the implant (since x mm of blood vessel growth toward the surface fills a greater volume of scaffold than it would nearer the center).

The phase of cell proliferation begins early on at around 24-48 hours, peaking at around 2-3 weeks. Tissue remodeling begins from around 1-2 weeks. Near complete degradation of the scaffold and tissue regeneration is achieved within 4 weeks.

### Example 2

### Freeze-Sublimation Methods for Constructing Gradient Scaffolding with Varied Pore Diameter

### Preparation of Slurry

Extracellular matrix components, such as, for example, microfibriallar, type I collagen, isolated from bovine tendon (Integra LifeSciences) and chondroitin 6-sulfate, isolated from shark cartilage (Sigma-Aldrich), 10% (w/w) are combined with 0.05M acetic acid at a pH ˜3.2 are mixed at 15, 000 rpm, at 4° C., then degassed under vacuum at 50 mTorr.

### Varying Pore Diameter

The suspension is placed in a container, and only part of the container (up to 10% of the length) is submerged in a supercooled silicone bath. The equilibration time for freezing of the slurry is determined, and the freezing process is stopped prior to achieving thermal equilibrium. The container is then removed from the bath and the slurry is then sublimated via freeze-drying (for example, VirTis Genesis freeze-dryer, Gardiner, N.Y.). Thus, a thermal gradient occurs in the slurry, creating a freezing front, which is stopped prior to thermal equilibrium, at which point freeze-drying is conducted, causing sublimation, resulting in a matrix copolymer with a graded average pore diameter field.

In another method, the suspension is placed in a container, on a freezer shelf, where a graded thermal insulation layer is placed between the container and the shelf, which also results in the production of a gradient freezing front, as described above. The graded thermal insulation layer can be constructed by any number of means, including use of materials with varying thermal conductivity, such as aluminum and copper, or aluminum and plexiglass, and others.

In one embodiment, the container is a multicomponent mold, containing removable elements. In one embodiment, removal of these elements creates tunnels within the frozen slurry. In another embodiment, the removable elements have conical shape, such that in one embodiment, the tunnel diameter narrows the further the distance is from the periphery of the scaffold.

In one embodiment, the surface of the mold creates indentations and channels in the frozen slurry, thereby creating surface folds of desired geometry and distribution across

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A solid, biocompatible scaffold for implantation, comprising surface folds, wherein said surface folds increase said scaffold surface area to volume ratio (SA/V), by at least 20%.

2. The scaffold of claim 1, wherein said folds vary in terms of their depth, which may range from 1 mm-10 cm, their diameter, which may range from 1 mm-5 cm, or a combination thereof, within said scaffold.

3. The scaffold of claim 1, wherein said folds form a channel within said scaffold.

4. The scaffold of claim 3, wherein said channel may be oriented along an axis.

5. The scaffold of claim 3, wherein the diameter of said channel is greater at a point more proximal to the scaffold surface, than to its core.

6. The scaffold of claim 1, wherein said folds are randomly distributed throughout said scaffold.

7. The scaffold of claim 1, wherein said scaffold comprises at least one polymer.

8. The scaffold of claim 5, wherein said polymer comprises at least one synthetic or natural polymer, ceramic, metal, extracellular matrix protein or an analogue thereof.

9. The scaffold of claim 6, wherein said extracellular matrix proteins comprise a collagen, a glycosaminoglycan, or a combination thereof.

10. The scaffold of claim 1, wherein said scaffold is non-uniformly porous.

11. The scaffold of claim 10, wherein said scaffold varies in its pore diameter, from 0.75 to 3000 μm, its average pore size distribution, which may range from about 20 to 200 μm±1 to about 50 μm, its cross-link density.

12. The scaffold of claim 1, wherein said scaffold further comprises cells, extracellular matrix components, growth factors, cytokines, hormones, inflammatory stimuli, angiogenic factors, or a combination thereof.

13. The scaffold of claim 1, wherein the size and shape of said scaffold is a function of the tissue into which the scaffold is to be implanted.

14. The scaffold of claim 1, wherein said scaffold, when implanted, promotes angiogenesis within, or proximal to the scaffold.

15. The scaffold of claim 1, wherein said scaffold is comprised of a material whose stiffness is sufficient to resist compressive forces of tissue proximal to a site of implantation.

16. A process for preparing a solid scaffold comprising surface folds, the process comprising the steps of:
a. applying a polymeric suspension to a mold comprised of a conductive material, wherein a surface of said mold which is in contact with said suspension has numerous folds; and
b. subjecting the suspension-filled mold in (a) to conditions whereby said suspension is solidified.

17. The process of claim 16, wherein the mold is so constructed that the surface folds vary in depth, from 1 mm-10 cm, and diameter, which may range from 1 mm-5 cm, or a combination thereof, within said scaffold.

18. The process of claim 16, further comprising the step of super-cooling the suspension-filled mold in (a), at a constant temperature, for a period of time until said suspension is solidified, whereby ice crystals are formed in said solidified suspension, said crystals being oriented perpendicular to an edge of said scaffold

19. The process of claim 18, further comprising exposing the solidified suspension or a portion thereof, to conditions which enable sublimation in the exposed region, whereby pores are formed which are perpendicular to an edge of said scaffold.

20. The process of claim 18, wherein the pores form a channel.

21. The process of claim 18, wherein the pores are oriented along an axis.

22. A scaffold prepared according to the process of claim 16.

23. A method of organ or tissue engineering in a subject, comprising the step of implanting a scaffold of any one of claim 1-15 or 22 in said subject.

24. The method of claim 23, further comprising the step of implanting cells in said subject.

25. The method of claim 24, wherein said cells are seeded on said scaffold.

26. The method of claim 25, wherein said cells are seeded at the periphery of said scaffold.

27. The method of claim 25, wherein said scaffold is cultured for a period of time prior to implantation in said subject.

28. The method of claim 25, wherein said cells are stem or progenitor cells.

29. The method of claim 25, wherein said engineering is of an organ or tissue comprised of heterogeneous cell types.

30. The method of claim 25, wherein said tissue is breast tissue or skin.

31. The method of claim 25, wherein said method is utilized in wound healing.

32. A method of organ or tissue repair or regeneration in a subject, comprising the step of implanting a scaffold of any of claim 1-15 or 22 in said subject.

33. The method of claim 32, further comprising the step of implanting cells in said subject.

34. The method of claim 32, wherein said cells are seeded on said scaffold.

35. The method of claim 34, wherein said scaffold is cultured for a period of time prior to implantation in said subject.

36. The method of claim 33, wherein said cells are seeded at the periphery of said scaffold.

37. The method of claim 33, wherein said cells are stem or progenitor cells.

38. The method of claim 33, further comprising the step of administering cytokines, growth factors, hormones or a combination thereof.

39. The method of claim 32, wherein said engineering is of an organ or tissue comprised of heterogeneous cell types.

40. The method of claim 32, wherein said tissue is skin or breast tissue.

41. The method of claim 32, wherein said method is used in wound healing
